(19) United States
(12) Patent Application Publication
(10) Pub. No.: US 2025/0212845 A1
(43) Pub. Date: Jul. 3, 2025

(54) SYSTEM AND METHOD FOR DETECTING LAMENESS IN CATTLE

(71) Applicant: Viking Genetics FMBA, Randers SØ (DK)

(72) Inventors: Jan Lassen, Randers SØ (DK); Søren Borchersen, Horsens (DK)

(73) Assignee: Viking Genetics FMBA, Randers SØ (DK)

(21) Appl. No.: 18/848,248

(22) PCT Filed: Mar. 27, 2023
(86) PCT No.: PCT/EP2023/057881

§ 371 Date: Sep. 18, 2024

(30) Foreign Application Priority Data
Mar. 25, 2022 (EP) 22164464.4

(51) Int. Cl. A01K 29/00; A01K 11/00; G06T 7/10; G06V 10/26; G06V 10/82; G06V 40/10

(52) U.S. Cl. CPC A01K 29/005; A01K 11/004; G06T 7/10; G06V 10/26; G06V 10/82; G06V 40/10; G06T 2200/04; G06T 2200/08; G06T 2207/10028; G06T 2207/10048; G06T 2207/20081; G06T 2207/20084

(57) ABSTRACT

The present disclosure relates to detection of lameness in bovine subjects, especially dairy cattle, in a shed environment based on vision technology, in particular 3D imaging. A first embodiment relates to a method for detecting lameness in bovine subjects, the method comprising the steps of acquiring at least one 3D image of the back of a bovine subject, extracting data representing the spine of the bovine subject, determining at least one curvature of at least one preselected part of the spine, and detecting lameness and/or determining a degree of lameness of the bovine subject by correlating said curvature(s) with at least one reference value, such as at least one predefined threshold value.

The present disclosure relates to detection of lameness in bovine subjects, especially dairy cattle, in a shed environment based on vision technology, in particular 3D imaging.

### BACKGROUND OF THE INVENTION

When running a large agriculture with a large number of individual animals it is a challenge to constantly be aware of each and every animals well-being. The wellbeing of the bovine subject is very important, both from the humane perspective of the farmer having the responsibility for the well-being of these animals, but also since the healthy, well-fed animals ensures a greater productivity and is of greater value to the farmer. Hence, it is of great interest to frequently monitor the well-being of a bovine subject.

Locomotor diseases causing lameness are considered as one of the most serious welfare issues for dairy cattle and cause increased costs for dairy farmers. Lameness is not a disease in itself but indicates pain or discomfort for the cow when moving. Lameness is characterized by a change in gait or an irregularity of the walking pattern. Lameness is typically caused by claw and/or leg disorders indicating the attempt of the cow to reduce the amount of weight bearing on the affected limb(s). Therefore, lameness is considered to be indicator of an underlying problem that may cause pain to the cow. Lameness is associated with lower feed intake, reduced milk production and impaired reproduction, and may lead to early culling. Hence, reduction of a cow's mobility impacts the overall health and welfare and should therefore be avoided. The majority of lameness cases in dairy cattle are related to lesions of the claws, infectious or non-infectious that induce the pain.

Subjective methods are currently used for assessing lameness of cows on farms, and results are typically provided in the form of numerical rating scores. Such subjective assessment typically rates individual cows regarding the presence or absence of certain behaviours and postures related to gait. These scoring systems focus primarily on locomotion or gait associated with the cow's degree of reluctance of bearing weight on the affected limb(s). The number of scoring categories typically range between two and five.

An example of a widely used subjective scoring system for assessing lameness is the Sprecher system, which is a five-point scale system ranging from 1 (“normal”) to 5 (“severely lame”). It is widely used due to its simplicity and the observation of the presence of behaviours, for example an arched back when standing and walking. An advantage of the Sprecher system is that it is non-invasive and can easily be applied under farm conditions. The Sprecher system allows people like dairy farmers and their employees, veterinarians, hoof trimmers and advisors to subjectively perform the lameness assessment for use in herd management. However, subjective scoring systems, like the Sprecher system, suffer from lack of precision, and they are typically only reliable for cows that are either moderately lame, lame or severely lame.

### SUMMARY OF THE INVENTION

Preventing lameness helps to optimize milk production, improves conception rates and animal welfare and reduces treatment costs and antibiotic use. Consequently, it lowers stress level in both, cows and dairy farmers. The purpose of the present disclosure is therefore to provide a system and a method for objectively detecting lameness and/or determining a degree of lameness of cows based on imaging of the cows, preferably exclusively based on imaging of the cows and preferably 3D imaging acquired from above the cows.

One aspect of the present disclosure therefore relates to a method for detecting lameness in an animal, typically a bovine subject, for example a bovine subject with known breed. The method comprises the step of acquiring at least one image, preferably 3D image, of the bovine subject, preferably of the back of the bovine subject. It has previously been shown that the back of the bovine subject carries a lot of information of the specific animal.

The method may further comprise the step of extracting data from said at least one image, preferably data relating to the topology and/or topography of the back of the bovine subject, for example extracting data representing the spine of the bovine subject, the spine may for example be represented by a line of local maxima along the back of the bovine subject.

At least one curvature of at least one preselected part of the spine can then be determined. Lameness can then be detected by correlating said curvature(s) with at least one reference value, such as at least one predefined threshold value. Additionally or alternatively the degree of lameness can be determined, for example based on the difference between the curvature(s) and corresponding reference value(s).

Alternatively and/or additionally lameness of the bovine subject can be detected by extracting data in the form of contour points, from said at least one 3D image, relating to the topology of the back of the bovine subject, detecting lameness and/or determining a degree of lameness of the bovine subject by correlating said contour points against a reference model.

The present inventors have realized that lameness of a bovine subject correlates with the 3D shape, i.e. topology and/or topography, of the back of the bovine subject. I.e. parameters such as age of the bovine subject, lactation, etc., can be left out of the estimation. This makes it possible to detect lameness of a bovine subject based on 3D images alone acquired from above such that the topology of the back of the bovine subject can be extracted from the 3D image. This makes it also possible to install the presently disclosed system in a stable, a cowshed, or the like and acquire images of the bovine subjects from above, for example while the bovine subjects are eating or while they pass through a lock, for example on their way to or returning from milking.

In the presently disclosed approach it has been shown that all it requires is a reference model and/or merely one or more curvature thresholds, possibly for the specific breed of the bovine subject and/or for the specific individual animal. It has also been shown that only a very limited number of topology predictors extracted from the acquired 3D image is enough to accurately detect lameness and/or the degree of lameness of the bovine subject. This eliminates the need for subjective scoring of each animal in the herd. This further allows for the continuous monitoring of the well-being of the bovine subjects on a day-to-day basis and even multiple times every day.

In particular the inventors have realized that the shape of the spine and/or contour points extracted from 3D image data of the back of the bovine subject correlates very good with lameness of the bovine subject, in particular dairy cattle. Hence, data can advantageously be extracted in the form of a discrete number of contour points from said at least one 3D image. The contour points can for example be extracted relative to a line of local maxima along the back of the bovine subject, typically defined as the spine of the bovine subject defining a longitudinal direction in the 3D image of the bovine subject. Possibly only data representing the spine is necessary to extract in order to detect lameness and/or a degree of lameness of the bovine subject.

The present disclosure further relates to a system for detecting lameness in bovine subjects, comprising an imaging system configured to acquire at least one 3D image of the back of a bovine subject, a processing unit configured for acquiring at least one 3D image of the back of the bovine subject, extracting data representing the spine of the bovine subject, determining at least one curvature of at least one preselected part of the spine, and detecting lameness of the bovine subject by correlating said curvature(s) against at least one predefined threshold value.

The presently disclosed system and method for lameness detection can completely eliminate the need for individually and subjectively handling each animal in the process of detecting and assessing lameness which enables the possibility of frequent monitoring of each individual animal in a large herd. For example the bovine subject may be walking through a lock/narrow aisle connecting a shed resting area with a feeding and/or milking area. The aisle may be so narrow that it only allow passages of one animal at the time. One or more images of the back of the bovine subject may then be acquired by one or more cameras placed above the aisle. The camera(s) may be of any type of camera providing 3D information, such as, but not limited to, a time of flight (ToF) camera, stereo camera, structured light camera, light-field camera, or a combination thereof. The imaging system may comprise a 2D camera and a depth sensor. The imaging system may be configured for acquiring topographic images.

As also disclosed in WO 2017/001538 by the same applicant such cameras can also be installed over the feeding area and used for acquiring 3D images of the bovine subjects. The individual bovine subject can be identified by means of the 3D images such that each bovine subject also can be monitored with regard to lameness in the stable.

The possibility that the bovine subject may be walking while acquiring an image allows for a frequent monitoring of lameness of the bovine subject, as this can happen without interfering with the daily life of the bovine subjects. This frequent monitoring of lameness of the bovine subject, further allows the farmer to gather data for statistics on lameness and/or the degree of lameness of bovine subjects in a herd. This data can be used in mathematical modelling of the bovine subjects in order to differentiate short-term and long-term fluctuations and changes. The short-term fluctuations may be due to inflation and/or feed content of the bovine subject and/or oedema in the bovine subject. Long-term changes however reflect for example weight changes of the body of the bovine subjects, such as
changes in muscle and fat amount and/or
distribution and/or
the growth of the skeleton in the case of a not fully grown animal, and/or
the growth of a foetus in the case of a pregnant animal and/or
the event of giving birth in the case of a pregnant animal.

Another aspect of the present disclosure relates to detection of lameness based on artificial intelligence, i.e. where a model has been trained to detect lameness preferably determine a degree of lameness, for example based on experts labelling data and/or labelling actual bovine subjects.

One embodiment relates to a method for detecting lameness in a bovine subject, the method comprising the steps of acquiring a plurality of range images of the back of at least one bovine subject. The range images are preferably acquired from above the at least one bovine subject. Image processing can then be applied to the range images to generate at least one sequence of 3D images of a preselected bovine subject. And then detecting lameness and/or determining a degree of lameness of the preselected bovine subject by modelling the at least one sequence of 3D images against a trained reference model employing artificial intelligence (AI).

A further embodiment relates to a system for detecting lameness in bovine subjects, comprising an imaging system configured to acquire a plurality of range images of the back of a bovine subject, and a processing unit configured for executing the presently disclosed methods.

I.e. the expert knowledge of for example a veterinarian can almost be brought into the stable and integrated in a vision system that acquires range images from above the bovine subjects, for example each time they walk towards milking, such that they can be continuously surveyed.

The presently disclosed methods can advantageously be computer implemented. The present disclosure also relates to a system for detection of lameness of a bovine subject, comprising a computer-readable storage device for storing instructions that, when executed by a processor, performs the methods disclosed herein.

The presently disclosed methods may be implemented as one or more separate computer programs but may also be implemented as a plugin to an existing service running on a device or online.

The present disclosure therefore also relates to a computer program having instructions which when executed by a computing device or system causes the computing device or system to perform the detection of lameness as disclosed herein. Computer program in this context shall be construed broadly and include e.g. programs to be run on a PC or software designed to run on smartphones, tablet computers or other mobile devices. Computer programs and mobile applications include software that is free and software that has to be bought, and also include software that is distributed over distribution software platforms or running on a server.

### DESCRIPTION OF THE DRAWINGS

The present disclosure will in the following be described in greater detail with reference to the drawings. The drawings are exemplary and are intended to illustrate some of the features of the present method and system and are not to be construed as limiting to the present disclosure.

FIG. 1 illustrates the contour lines of the back of a bovine subject corresponding to height drops relative to the spine level.

FIG. 2 is a schematic view of an embodiment of the process of generating the contour plots of the back of the bovine subject.

FIG. 3 is a schematic view of the canalizing guidance of animals through a passage/lock/narrow aisle from one shed area to another, on the way to milking or on the way back from milking.

### DETAILED DESCRIPTION OF THE INVENTION

### Topographical Approach

A stated above one embodiment relates to a method for detecting lameness in bovine subjects, the method comprising the steps of
acquiring at least one 3D image of the back of a bovine subject, and
extracting data representing the spine of the bovine subject,
determining at least one curvature of at least one preselected part of the spine, and
detecting lameness and/or determining a degree of lameness of the bovine subject by correlating said curvature(s) with at least one reference value, such as at least one predefined threshold value.

and/or
extracting data in the form of contour points, from said at least one 3D image, relating to the topology of the back of the bovine subject,
detecting lameness and/or determining a degree of lameness of the bovine subject by correlating said contour points against a reference model.

The extracted data may comprise at least 50 data points, preferably at least 100 data points. The reference model may comprise information of the topology of the back versus the degree of lameness, for example in relation to the breed of said bovine subject.

The normal meaning of the term “topography” is that it is the study and description of the physical features or shape of an area, for example its hills, valleys, or rivers, or the representation of these features on maps, i.e. usually used with geography. In this case the topography of the back of the bovine subject is therefore understood as the shape of the back of the bovine subject, i.e. the 3D shape with whatever “hills” and “valleys” that appear on of back of the bovine subject.

The term “topology” as used herein is the anatomy of a specific bodily area, structure, or part, in this case typically the back of the bovine subject. A bovine subject's anatomy is the structure of its body, e.g. the outside shape of the body.

The normal procedure when subjectively assessing and scoring lameness by manual observation of the individual cow is that if the cow stands and walks with a flat back posture and the movement is smooth and fluid with normal gait, then the cow is not lame, whereas if the cow stands with a level-back posture but develops an arched back posture while walking, the cow is considered to be mildly lame. The Sprecher scoring system continues to level 5 where the cow has an extreme arched back when standing and walking, with and obvious joint stiffness where the head obviously bobs as sore limb/hoof makes contact with the ground.

However, based on massive 3D imaging data acquisition from five herds of dairy cattle the inventors have realized that it does not make sense to speak of a “flat back” of a cow, because all cows have curved backs and the curvature varies across the longitudinal extension of the back. The inventors have also surprisingly discovered that there is a variation in the variation of the curvature across the back. In order to detect small indications of lameness it is therefore crucial to have a suitable reference value(s) and/or a suitable reference model, possibly specific to the individual herd, breed and/or bovine subject.

Hence, the reference model may comprise information of the topology of the back versus the degree of lameness, for example in relation to the breed of said bovine subject. Additionally and/or alternatively the reference value(s) and/or the reference model can be specific to said bovine subject. With the possible image acquisition setup outlined herein the reference value(s) and/or the reference model can advantageously be generated from previously acquired 3D images of the back of said bovine subject, e.g. acquired over a time period of at least two days, more preferably at least five days, even more preferably at least one week, yet more preferably at least two weeks, most preferably at least four weeks.

The curvature of the spine, or only a preselected part of the spine, is preferably defined as only the vertical curvature of said preselected part of the spine, i.e. it is the rate of vertical change along a longitudinal extension of the cow, i.e. between head and tail. The curvature of a preselected part of the spine is then preferably determined by calculating the slope of said preselected part of the spine.

The inventors have realized that the rear part of the spine is more important for detecting and assessing lameness, i.e. by exclusively looking at only at least one preselected part of the spine which is a rear part of the spine, i.e. towards the tail of the bovine subject. The rear part can be defined as the part between the tail part of the spine and a middle part of the spine.

The inventors have further realized that a lot of information is hidden in the local curvature of the spine of the bovine subject. Hence, at least part of the spine can be divided into subsections, wherein the curvature of each subsection is determined, such as at least 2, 3, 5, 7, preferably at least 8 or 9 or at least 10 subsections. In that regard a reference value can be provided for each subsection, or at least a plurality of said subsections. An then further the detection of lameness may correlate with a predefined difference between the curvature(s) and their corresponding reference value. Additionally the degree of lameness may correlate with the amount of the difference between the curvature(s) and the corresponding reference value.

In the preferred embodiment the extracted data used for correlation against the reference model comprises a discrete number of contour points in order to simplify the calculation, for example a discrete number of contour points defining the spine. For example less than 1000 contour points, preferably less than 500 contour points, even more preferably less than 250 contour points, most preferably less than or equal to 100 contour points, such as 50 or only 25 or even 12 contour points.

It can be shown that if appropriately selected, only 100 or 50 contour points extracted from a 3D image of the bovine subject's back can be sufficient to detect lameness in a bovine subject.

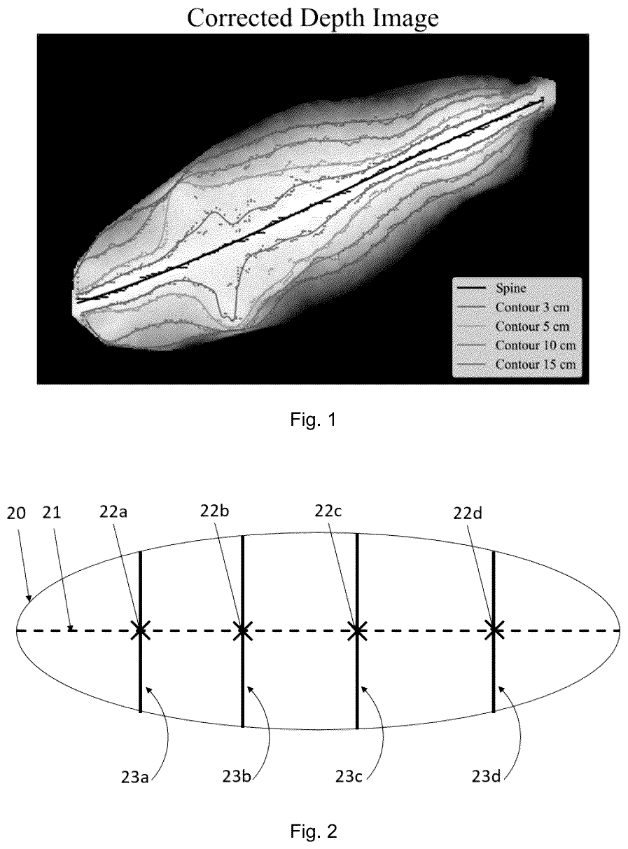

The spine of the bovine subject is preferably located in the at least one 3D image of the back of the bovine subject. The spine of the bovine subject may be defined as a through-going line of local height maxima as exemplified in FIG. 1. Consequently the spine of the bovine subject can be used to define a longitudinal direction in the 3D image of the bovine subject.

In one embodiment data is extracted from the 3D image by contour plotting the back of the bovine subject thereby generating contour lines relative to the spine. A contour line may be based on the relative drop of height relative to the spine height at a given position along the spine, i.e. a contour line connects contour points of equal elevation. Hence, a single contour point of a contour line may be defined as a predefined decrease of height on the back of the bovine subject relative to the height of the spine, wherein the decrease of height relative to a point on the spine of the bovine subject is found along a line perpendicular to the spine, as exemplary illustrated in FIG. 2.

As stated above the inventors have realized that the lameness, and optionally the degree of lameness, of the bovine subject can be predicted with only a limited number of data points extracted from the 3D image. In one embodiment the extracted data comprises less than 20 contour points, such as less than 10 contour points, such as between 1 and 10 contour points, such as less than 7, 6 or 5 contour points, such as 4 contour points, selected from the spine, and/or less than 20 contour points, such as less than 10 contour points, such as less than 7, 6 or 5 contour points, such as between 1 and 10 contour points, such as 4 contour points, selected from each of less than 10 contour lines, such as less than 7, 6 or 5 contour lines, such as between 1 and 10 contour lines, such as 3 contour lines, relative to the spine. The contour lines are preferably generated at discrete intervals at a height less than or equal to 15 cm, preferably less than or equal to 10 cm, relative to the height of the spine. For example contour lines at 2.5 cm, 5 cm and 10 cm relative to the spine.

The realization that lameness detection is possible based on 3D imaging of the bovine subject's back makes it possible to acquire one or more images while the bovine subject is moving. Hence, in one embodiment of the present disclosure the bovine subject is moving during acquisition of said at least one 3D image. More than one 3D image may be acquired of the bovine subject. Hence, said at least one 3D image is preferably based on a plurality of 3D images, and these may be acquired while the bovine subject is moving. Consequently, the data analysis may be based on a median image of two or more images.

An example of contour plotting is illustrated in FIG. 1 in which each line indicates a contour plot of the back of a cow. The middle longitudinal line is formed by local maxima of the height in the image and defines the spine of the bovine subject. In one embodiment contour plotting are lines and/or points along the back of the bovine subject all corresponding to a specific amount of height drop relative to the spine in that particular area.

One possible strategy to develop such a contour plot is illustrated in FIG. 2, wherein the body of the bovine subject is represented by an ellipse 20. The spine is found along the back of the bovine subject as the tallest part of the central area of the back, i.e. local height maxima, when going along a central line connecting the neck to the tail. The position of the spine, the spine axis, is represented by the dashed line 21 in FIG. 2. Along the spine axis 21 a given number of points of interest is selected. In FIG. 2, four points have been selected and are denoted 22a-d and are marked with each their cross along the spine. From a selected point of the spine, say 22a, a line is drawn perpendicular to the axis of the spine, this perpendicular axis is denoted 23a in FIG. 2. From the point of interest on the spine, 22a, the perpendicular line 23a is followed along one direction towards the edge of the bovine subject, for example towards the right of the bovine subject. Once a height drop of X cm relative to the height of the spine in 22a is reached, this point is noted in the database. X is referring to a real number. Next this process is repeated as one goes along 23a toward the opposite side of the bovine subject, which could hence be the left of the bovine subject, and the same level of height drop of X cm is noted on this opposite side of the spine. This process is repeated for all points of interest 22b-d along the spine 21 and along the respectively perpendicular lines 23b-d. Once all the points representing a given height drop of X cm relative to the points of interest 22a-d along the spine axis 21 has been located, a line is fitted to best describe the position of said points. This fitted line now represents the contour line of the relative height drop of X cm relative to individual points along the spine. If more contour lines for different values of height drops are desired the process may be repeated for other values of relative height drop relative to the height of each point along the spine. Alternatively, all relevant contour points along a given perpendicular line may be found before moving on to the line perpendicular to the next point of interest along the spine. These relevant contour plots may for example be X cm, Y cm, Z cm and T cm, where X, Y, Z and T referring to real numbers. In FIG. 1 for example, the values of X, Y, Z and T are 3, 5, 10 and 15 cm, respectively, each resulting in their respective contour line.

The illustration of FIG. 2 is a simplified illustration for the purpose of illustrating the described strategy to develop a contour plot in the simplest possible way. Hence, the bovine subject is illustrated as an ellipse for simplicity of the illustration only. In other words, the bovine subject's body shape is not considered an ellipse by the strategy of developing a contour plot. In the true data handling process, as in FIG. 1, the spine is not necessarily a perfect straight line as the bovine subject has an irregular shape and not an ellipse. Hence, the perpendicular line of a given point along the spine may be estimated based on a number of neighbouring point on the spine, to the point of interest. These points may for instance be fitted to a straight line and the perpendicular line is determined based on this fitted line. Hence, the best estimate for a perpendicular line for a given point of an irregular line is made in the case of treating true data.

Hence, the contour plots may be based on a discrete number of points and a best fit to these points. In this manner the contour plots reflects a drop in height relative to the spine in every position along the back of the bovine subject rather than the contour plots with a fixed, same reference for all point of the contour plot. Hence, in this embodiment a single contour point of a contour line reflects a specific decrease of height on the back relative to the height of the spine along a line perpendicular to the spine of the bovine subject, passing trough said contour point, so that the basis of the contour plot is not an ultimate height relative to a given fix point, but every contour point is calculated relative to each their separate point of reference along the spine of the bovine subject. This further means that the contour plotting of the bovine subject's back may happen along the length of the back of the bovine subject, which in other words is along the spine of the bovine subject. In the preferred embodiment the lines connecting the individual point of the topography of the back is made as a best fit to the points representing a given height drop, and hence the fitted lines reflects the best fit for a contour line.

In an embodiment of the present disclosure, more than one image is acquired of the back of the bovine subject. This may be multiple images of the bovine subject while it is walking below the camera or multiple image of the bovine subject standing still. If the bovine subject is moving the images may be required for as long as the bovine subject has either a part of the body inside the imaging frame of the camera. Alternatively the images may only be acquired for the period of time of which the bovine subject has its entire body inside the imaging frame of the camera. Yet another alternative is that the camera may only acquire images for a given, pre-set period of time or the camera may require a pre-set number of images.

In the preferred embodiment the data analysis is based on a median image of two or more images. Hence, all or some of the acquired images should be combined to generate an average of the shape of the back of the bovine subject. The term “median image” refers to the average image, generated as an average of the information of the topography and/or anatomy of the back of the bovine subject in all collected images. One advantage of using such a median image is that the movement of the back of a bovine subject which is walking during image acquisition, can be smoothed out in the median or average image so that the walking induced variations in the topography can be eliminated.

In an embodiment, the bovine subject is standing still during the image acquisition. Preferably, the method will be compatible with both moving animals and animals which are standing still. Preferably, the imaging system is configured to acquire the image while the bovine subject is walking. In an embodiment, the imaging system is configured to acquire the image while the bovine subject is standing still.

In the preferred embodiment the acquisition of the at least one image of the back of the bovine subject is carried out at least once, preferably multiple times, per day to capture time- and condition-specific variations.

Preferably, all acquired data is saved in a database. The data can then be accessed at later times and it is furthermore possible to plot a time dependent plot of the development of the curvature of the spine and/or curvatures of subsections thereof, which may help in the process of finding animals which are not well. The method may hence be combined with methods for pattern recognition and/or machine learning, to make early state detection of lameness. In an embodiment, the process of monitoring and modelling the curvature changes and/or contour changes of the bovine subject is based on a large number of measurements acquired over an extended period such as multiple days, preferably multiple weeks and more preferably multiple months. Preferably, these data also contains multiple data points per day for each day over the acquisition time and in the preferred embodiment, this data is supported by the time schedule of the feeding and/or milking event of the bovine subject. As the process of inflation as well as the feed intake is strongly dependent on the time since the last feeding, this data strongly supports the modelling of short-term fluctuations if the data is accompanied by data regarding the amount of time since the last feeding and/or milking.

When evaluating the cow's timely development it may be of interest to evaluate short term variations and long term variations separately. Preferably, the data history is used to generate a model to divide for example the curvature variations into contributions of at least long-term variations and short-term fluctuations. More preferably, the measurements are used to mathematically model short-term fluctuations and long-term variations. Hence the model may subdivide variations in the curvatures into short-term fluctuations and long term changes. In an embodiment of the present disclosure the short-term fluctuations in the curvatures are changes during the day or on a day-to-day basis. These short-term fluctuations may be ascribed to inflation and/or feed content of the intestinal system and/or oedema of the bovine subject. The long-term changes in the curvatures may be considered gradual changes over a period of at least several days, preferably over several weeks, more preferably over several months. These long-term variations in the curvatures can be key to determine early signs of lameness.

### Spatial and Temporal Correlation Approach

This approach relies on recoding a time series of 3D data of the bovine subjects to detect lameness of the bovine subjects based how the bovine subject looks and how it moves, and employ a trained AI model to detect lameness.

One embodiment relates to a method for detecting lameness in a bovine subject, the method comprising the steps of acquiring a plurality of range images of the back of at least one bovine subject, wherein the range images are acquired from above the at least one bovine subject, image processing the range images to generate at least one sequence of 3D images of a preselected bovine subject, and detecting lameness and/or determining a degree of lameness of the preselected bovine subject by modelling the at least one sequence of 3D images against a trained reference model employing artificial intelligence (AI).

The range images can be 3D images that are directly acquired or it can 2D image data comprising depth information, e.g. from a RGB 2D camera combined with depth information from an infrared sensor, like a time-of-flight system, as known from the Kinect system. 2D image data combined with depth information can be processed to become 3D images.

The plurality of range images may be a time series of range images, and/or the sequence of 3D images of the preselected bovine subject may be a time series of 3D images.

The outcome of the presently disclosed approach can come from a step of labelling the preselected bovine subject as “not lame”, “possibly lame” or “moderately lame”, “lame” or “severely lame”, i.e. after AI modelling. Supplementary a step of labelling the preselected bovine subject as “lame” with a degree of lameness as an integer between 1 and 5 after detection of lameness thereby determining a degree of lameness, like the Sprecher score.

In order to provide a good temporal correlation, the range images are preferably acquired with a high frequency, i.e. the range images may be acquired with a frame rate of at least 10, or 15 fps, preferably at least 20 frames per second (fps), more preferably at least 25 fps, or even at least 30, 40 or at least 50 fps.

In order to instigate lameness detection it would be good that the bovine subject is actually moving, because it might happen that the animals stop during the passage og the imaging unit. Hence, the sequence of 3D images, typically selected from the acquired range images, comprise the preselected bovine subject walking forwardly. Correspondingly it may be advantageous for lameness detection that the selected sequence of 3D images for use in the modelling comprise the preselected bovine subject walking forwardly over a minimum distance of at least 1 meter, or at least 2 m, preferably at least 3 m, or 4 m, more preferably at least 5 m, most preferably at least 7 m, such as between 5 and 10 m. Similarly the sequence of 3D images selected for modelling comprise the preselected bovine subject during a minimum period of at least 1 second, or at least 2 second, preferably at least 3 seconds, more preferably at least 5 seconds, most preferably at least 7 or 10 seconds, such as between 5 and 10 seconds.

As stated above the range image acquisition includes acquisition of infrared (IR) 2D image data, typically in order to provide depth information. However, the inventors have realized that if IR data is in 2D, this additional 2D IR image data can be an extra modality fed into the AI model to enhance detection of lameness. Hence, the image processing may comprise the step of generating at least one sequence of 2D IR images of the back of the preselected bovine subject corresponding to the at least one sequence of 3D images.

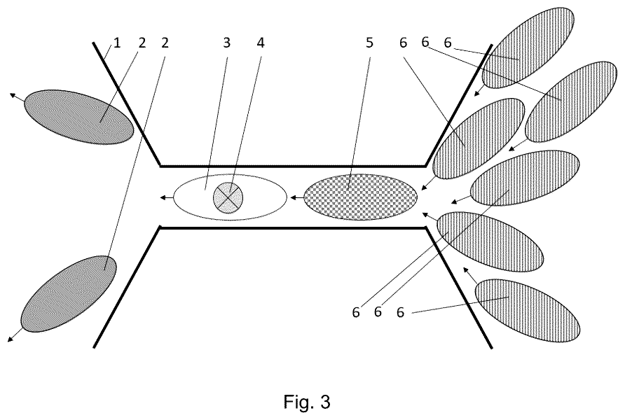

Preferably the range images are acquired from above a passage where through a plurality of bovine subjects are passing through, such as passing through at least once daily, e.g. as illustrated in FIG. 3. Correspondingly the step of identifying the preselected bovine subject by means of at least one ID reader located near one or both ends of the passage. Actually this identification may be part of selecting the preselected animal, because the time instance of identification readout provides a reference to the time series of range images such that a relevant sequence of 3D images can be generated for the identified bovine subject to become the preselected bovine subject. Hence, the sequence of 3D images of the preselected bovine subject preferably corresponds to a temporal (window) selection of the plurality of range images selected such that only an identified/preselected bovine subject is present in the sequence of 3D images.

The image processing step preferably comprise background removal to isolate the bovine subject from the surroundings of the range images. This background removal can also be part of the segmentation process, because for example known background elements can be segmented, identified and removed from the 3D image data.

The image processing preferably also comprise correction of perspective of the range images and/or the 3D images, in order to provide a good reference coordinate system for the 3D setup. This can be provided by image processing methods known in the art.

The image processing advantageously comprises the step of segmenting at least part of the acquired range images by means of for example instance segmentation in order to select a bovine subject among the segmented instances and generate said at least one sequence of 3D images of the preselected bovine subject. Instance segmentation can be provided by means of Mask-RCNN (Mask region-convolution neural network) which is an open source model which is a variant of a Deep Neural Network, and which can detect objects in an image and generate a high-quality segmentation mask for each instance. I.e. instance segmentation can provide a mask that describes/comprises/delimits the associated pixels for each bovine subject in the acquired range images. Other variants of instance segmentation neural networks exist, for example Cascade R-CNN and Mask2Former, that can also be applied here. In that regard the segmented bovine instance located closest to an ID reader and at the time of a corresponding ID reading can be associated with an identification of a segmented bovine subject, leading to a selection of the preselected bovine subject.

The presently disclosed approach may further comprise the step of generating point clouds of the preselected bovine subject based on the range images and the image processing, in particular the segmentation, e.g. point clouds can be generated based on the masks from the instance segmentation. The depth information can then turn the point clouds into real 3D point clouds with 3D coordinates in order to generate the at least one sequence of 3D images comprise point cloud coordinates representing the preselected bovine subject in the images, i.e. a sequence of 3D images with labelled point cloud data. And this can be combined with labelled 2D infrared data. Perspective correction can also be applied here. These data with several modalities can be input to the a trained machine learning model, e.g. a neural network, e.g. a convolutional neural network, in order to detect and/or characterize lameness of the bovine subject.

Hence, the sequence of 3D images used as input to the AI model comprise 3D point cloud data may represent movement of the preselected bovine subject in said sequence. And the sequence of 3D images used as input to the AI model may further comprise 2D IR data of the preselected bovine subject.

The (trained) reference model may comprises information of the topology of the back a bovine subjects versus the degree of lameness, for example in relation to the breed of said preselected bovine subject. The trained reference model is preferably trained to include a spatial correlation of the lameness in the sequence of 3D images and a temporal correlation of the lameness in the sequence of 3D images. The trained reference model may be specific to said preselected bovine subject, for example specific to the breed of said bovine subject.

The trained reference model may be trained with a degree of lameness as outcome. The trained model may be trained using a supervised learning approach, wherein the AI model is trained using labelled data and wherein the labelled data are obtained from expert veterinarians determining the degree of lameness of bovine subjects determined to be lame by the expert veterinarians. The trained reference model may be trained using a combination of self-supervised learning and semi-supervised learning, wherein self-supervised learning approach is provided to pre-train the AI model on unlabelled data, and then the pretrained AI model is fine-tuned on a smaller set of labelled data using semi-supervised learning

The trained reference model may be trained using machine learning, neural network, recurrent neural network, convolutional neural network, or any combination thereof.

The AI model MAY BE selected from the group of: machine learning, neural network, recurrent neural network, convolutional neural network, or any combination thereof.

The actual modelling preferably includes both spatial correlation and a temporal correlation of lameness in the sequence of 3D images with the trained reference model. I.e. the AI modelling becomes a sort of video prediction or a sequence prediction model that is capable of recognizing a pattern of movement of a bovine subject resulting from lameness when imaged from above while the bovine subject is moving, typically moving forward. Examples of sequence prediction models is multiscale 3D Resnet, like ST-3DGMR (Spatio-temporal 3D grouped multiscale ResNet network), that is readily available, or ViViT (video visin transformer), wherein spatio-temporal tokens from the sequence of 3D images of the bovine subject can be encoded by a series of transformer layers. Sequence prediction model can typically vary the temporal and spatial relationships independently, to handle the large amounts of data in video prediction.

The spatial and temporal modelling approach further relates to system for detecting lameness in bovine subjects, comprising an imaging system configured to acquire a plurality of range images of the back of a bovine subject, and a processing unit configured for executing the presently disclosed methods. The imaging system may be configured to acquire said range images while the bovine subject is standing in and/or walking through a lock and/or passage. The imaging system may be configured such that said range images are acquired from above the bovine subject thereby imaging said bovine subjects in a top-view. The imaging system may be configured to start acquisition of the range images when triggered by at least one bovine subject approaching and/or entering a lock/the passage. The imaging unit may comprise at least one RGB camera and a depth sensor, such as an infrared sensor for providing 2D infrared images. The imaging system may be configured to acquire the range images with a frame rate of at least 15 frames per second, preferably at least 20, more preferably at least 25 frames per second.

The system may comprise one more ID readers for reading an identification number of a bovine subject entering and/or exiting the lock/passage.

### Predictive Analysis

One of the advantages of an automatic bovine monitoring system which can be installed above a animal passage, is that the many animals can be monitored over a long time period to generate a large amount of 3D image data of the animals moving. Possibly acquired over weeks, months and even years. more than one year, can be available. This can be utilized for very early predictive detection of lameness, because bovine subjects that are detected as clearly lame, by the presently disclosed approach or by experts, such as a veterinarian, can be used as input to

In one embodiment only the most recent image data in a dataset is labelled by experts, or simply older cows with manually detected clear lameness, e.g. data from the last week, two weeks, or preferably three weeks, more preferably four weeks, most preferably two months. This labelled dataset, where each bovine subject has been assessed by an expert and categorized lame or not lame, or with a degree a lameness, can then be used as outcome for training a model based on earlier data of the same animals, e.g. data which is at least 1 month, or at least 2 or at least 3, preferably at least 4 months, more preferably at least 5 months earlier, most preferably at least 6 months earlier. E.g. a dataset may include data acquired over a time period of 12 months. The data for months 10-12 is labelled by experts, or the animals are manually evaluated, and used as outcome to train a model based on data for months 1-3 or 1-6, i.e. data acquired at least 4-6 months earlier than what has been assessed by experts. This may lead to the discovery of early predictors for lameness, predictors that are not even visible or known to the experts, thereby significantly improving the model.

### Passage

In one embodiment the bovine subject is walking during the image acquisition. The 3D image may hence be acquired while the bovine subject is moving, for example walking in a straight line. The ability to detect lameness information of a moving animal is a great advantage as it allows lameness detection and monitoring of a large number of animals in a row, and hence this eliminates the need for individually handling the individual animal. When animals are walking from a shed resting area to a feeding and/or milking area, or the opposite direction, the bovine subjects may be passing through a narrow aisle thereby only allowing one animal to pass at a time. By placing an imaging system above this narrow aisle the bovine subjects will walk by the area below the camera which can then capture an image of each individual animal as they are passing through the aisle. A great advantage hereto is that the bovine subjects will not be overlapping the view of each other, seen from the 3D camera from above. Hence, the bovine subjects are passing the narrow aisle and passing through the frame area of the 3D camera as they are being canalized or lead from for example a rest area to a feeding and/or milking area or the opposite direction. Another advantage of the narrow aisle is that it is possible to gain a good control the flow of animals.

An example of this process of leading the bovine subject trough a narrow aisle is shown in FIG. 3. Here the thick lines 1 illustrates the boundaries of the areas of which the bovine subjects can move. The boundaries may be a hence or a wall or similar enclosure mechanism. Each animal is, for simplicity of the figure, illustrated by an ellipse in FIG. 3. The arrow connected to each animal (ellipse in the figure) illustrates the direction of which the bovine subject is moving. The bovine subjects 2 to the left are the bovine subjects which have already passed the narrow aisle, the bovine subject 3 standing just below the 3D camera 4 is a bovine subject situated within the narrow aisle and currently being imaged by the 3D camera 4. The bovine subject behind 5 has just entered the narrow aisle and will be imaged as it soon passes through the area just below the 3D camera 4 hence, when animal 5 reaches the current position of animal 3. The bovine subjects to the right 6 all represent the bovine subjects still waiting to enter the narrow aisle to ultimately enter the area to the left of the aisle.

### Imaging Unit

The imaging unit comprises one or more cameras, preferably cameras adapted for range imaging, such as range cameras. There may also be several imaging units distributed along an bovine subject passage such that the bovine subjects can be imaged over a longer distance while moving. Examples of range imaging techniques applicable herein are time-of-flight, stereo triangulation, structured light, light-field imaging, e.g. 4D light-field imaging etc. The imaging unit may also be configured for acquiring topographic images. Each range camera may be provided with a depth sensor and a 2D camera, such as a RGB camera, for example as known from the Kinect system. The imaging unit may comprise at least one infrared (IR) sensor, such as an IR camera, preferably providing 2D IR data. Depth data can then be acquired by means of the IR signal.

The imaging unit is preferably configured for acquiring images over a time period with acquisition of multiple frames per second (fps), such that the output is more or more video sequences. E.g. at least 5, 10, or 15 fps, preferably at least 20 fps, most preferably at least 25 fps or even at least 30 fps, preferably both RGB, depth and IR, such high fps that video sequences comprises both 3D data and 2D IR data.

The time period for video acquisition may be triggered by detection of bovine subjects near the imaging unit, and video acquisition continues while one or more bovine subjects pass by. Subsequent image processing, e.g. by segmentation, can separate data such that video sequences of each bovine subject can be extracted. The presently disclosed approach is preferably configured such that a single bovine subject is imaged in a time period while passing the aisle/the imaging unit, of at least 1 second, preferably at least 2 seconds, more preferably at least 3 seconds, most preferably at least 5 seconds, or even 8 or 10 seconds, for example between 5 and 10 seconds. The bovine subject is preferably moving in the video sequence such that the movement dynamics of the single bovine subject is recorded in the video sequence.

Alternatively or supplemental the presently disclosed approach can be configured such that a single bovine subject is imaged over a predefined minimum distance to be certain that there is movement, preferably walking movement, in the images. E.g. a minimum distance of 2 m, or at least 3 m, preferably at least 5 m, most preferably at least 7 or even at least 10 m for example between 5 and 10 meters.

The imaging system typically includes at least one optical device, e.g. a camera, and in order to keep the at least one optical device clean the device may have to eventually be cleaned and/or possibly protected from the dirt of the shed environment. One possible solution is to use a protective cover keeping the dirt away from the actual optical device. In the preferred embodiment the at least one optical device is protected by a protective cover to prevent dirt from covering the functioning parts of the optical device, directly. In a further embodiment the cover of the at least one optical detector is cleaned using an automatized cleaning system such as an automatic windscreen wiper. Hence, if the optical device cover is getting too dirty for capturing the desired data the cover can get cleaned automatically by running the cleaning system such as an automatic windscreen wiper.

Another approach to keep the at least one optical detector clean is for example by having the at least one optical detector protected by a gate or a shutter only opening for a short period of time upon acquiring the data. Hence the detector will only be exposed for a very short amount of time upon data collection by the at least one optical detector. Thus, in one embodiment the at least one optical detector is protected by a gate or a shutter only opening for a short period of time upon acquiring the data. Hence, the gate or shutter will open briefly for image acquisition, and will then close again to avoid the optical device getting dirty. In the latter case the optical device will contain both a protective cover and a gate/shutter, the gate/shutter hence preventing the cover of the optical device to get dirty too quickly. To optimize the time of which the optical detector is exposed to collect data, hence to optimize the time of the shutter to open the shutter or gate of the system may include another feedback system, to determine when a bovine subject is standing in a proper position for data acquisition. This feedback system may be an independent system based on a sensor placed next to the 3D camera. The position of the bovine subjects may be determined from another detector and/or sensor not covered by a shutter or a gate and this determines when a bovine subject is in a proper position for imaging. When this happens the shutter will open and the range images will be taken after which the shutter closes immediately. It may also be possible to acquire a series of images before the shutter closes.

In a further embodiment the at least one optical detector includes a cleaning alert system sensing when the sensor needs to get cleaned and notify the user through said alert system such as a light turning on, a noise playing or a wireless signal transmitted to a computer. This is particularly useful if the device does not have an automatized cleaning system or if it is insufficient to fully clean the device. In this case the alert system will make the user aware that further cleaning of the device or the cover of the device is needed. The user might then manually clean the device or activate an automatized cleaning system. The cleaning process may then be automatic or manual. If the automated cleaning process is sufficient it may not be necessary to notify the user and this step might be left out as long as the system can efficiently self-clean.

### Identification

It is an advantage for the further data analysis and monitoring of the individual animal to know the identity of the specific imaged animal. Identification may be manual, for example by reading the identification number of the ear tags of the bovine subject, or electronic identification means such as radio frequency ID (RFID), by pattern recognition of the bovine subject, by a known, orders sequence of the bovine subjects or any other mean of identification. The bovine subject may be identified prior to the image acquisition or after the image acquisition.

If using subject specific reference it might be necessary to know the identity of the bovine subject in the image(s). But as shown in WO 2017/001538 it is possible to (uniquely) identify animals in a population of known animals based on images of the bovine subject's back. Hence, the presently disclosed approach may further comprise the step of identifying the bovine subject based on said at least one 3D image.

Identification of the animal may not be necessary in order to detect lameness. However, it is an advantage for the further data analysis and monitoring of the individual animal to know the identity of the specific imaged animal. Identification may be manual, for example by reading the identification number of the ear tags of the animal, or electronic identification means such as radio frequency ID (RFID), by pattern recognition of the animal, by a known, orders sequence of the animals or any other mean of identification. An identification (ID) reader, for example for reading of (RFID) ear tags, is typically located near the aisle, i.e. at the entrance and/or at the exit of the aisle, where the animals are supposed to pass one by one. Note that an entrance may be an exit and vice versa, dependent on which way the animals pass. Hence, the animal may be identified prior to the image acquisition, during the image acquisition, and/or after the image acquisition.

As shown in WO 2017/001538 it is possible to (uniquely) identify animals in a population of known animals based on images of the animal's back. Hence, the presently disclosed approach may further comprise the step of identifying the animal based on said at least one 3D image.

The system may also comprise ID tags. Such ID tags may be connected to animals to be identified. ID tags may be visual and/or electronic ID tags. Electronic ID tags may be electronic ear tags and/or electronic ID tags attached to an animal such as in a collar. A single animal may be marked with one or more ID tags such as at least one visual ID tag and/or at least one electronic ID tag. An example is at least one visual ear ID tag combined with at least one electronic ID tag in a collar. Another example is at least one visual ear ID tag combined with at least one electronic ear ID.

### Segmentation

A plurality of bovine subject may be passing through at the same time and the acquired 3D images may contain multiple animals. Hence, segmentation can advantageously be applied to isolate the individual bovine subject in the images, for example in the form of semantic segmentation to group pixels in a semantically meaningful way such that pixels belonging to different objects are grouped separately. However, preferably in the form of instance segmentation such that a label is assigned to each object in the images, such that instance segmentation can be provided of the individual bovine subject in the 3D images, preferably in the sequence of 3D images.

Instance segmentation typically includes object detection of all objects in an image in order to classify individual objects and localize each object instance using for example a bounding box, and then segmenting of each instance in order to classify each pixel into a fixed set of categories without differentiating object instances. In the present approach it is most important to classify the individual bovine subjects such that a sequence of 3D images comprising more than one bovine subject is segmented to separate the individual bovine subjects in to separate instances.

One example of instance segmentation is the known Mask-RCNN (Mask region-convolution neural network, it is a variant of a Deep Neural Network and detects objects in an image and generates a high-quality segmentation mask for each instance) that can provide a mask that describes the associated pixels for each bovine subject in the sequence of 3D images. Other variants of instance segmentation neural networks exist, for example Cascade R-CNN and Mask2Former, that can also be applied here.

### Breed

As used herein the bovine subject may be a bovine subject, preferably including both cows and bulls, whether adult or newborn animals. Consequently the breed of the bovine subjects may be selected from the group of: The Jersey breed, the Holstein breed, the Holstein-Friesian cattle population, Holstein Swartbont cattle population, the Deutsche Holstein Schwarzbunt cattle population, the US Holstein cattle population, the Red and White Holstein breed, the Deutsche Holstein Schwarzbunt cattle population, the Danish Red population, the Finnish Ayrshire population, the Swedish Red and White population, the Danish Holstein population, the Swedish Red and White population and the Nordic Red population.

### EXAMPLES

### Example 1

Data from more than 1000 dairy cows from five different herds were acquired over a time period in the form of 3D images. As indicated from the ratio between number of cows and number of images in Table 1 above, each cow was imaged many times during the time period.

TABLE 1
Data from five herds

| Herd | Number of cows | Number of images |
|---|---|---|
| 1 | 257 | 35810 |
| 2 | 162 | 21597 |
| 3 | 138 | 9029 |
| 4 | 311 | 41459 |
| 5 | 150 | 24115 |

After each milking a 3D image was acquired of the back of the cow (FIG. 1). The highest point of the back of the cow is the spine. Along the spine, 100 points are placed evenly. The 90 points towards the end of the back of the cow is used to generate 9 slopes. The slope from point 1 to 10 (slope1), from 11 to 20 (slope2), from 21 to 30 (slope3), from 31 to 40 (slope4), from 41 to 50 (slope5), from 51 to 60 (slope6), from 61 to 70 (slope7), from 71 to 80 (slope8) and from 81 to 90 (slope9). A negative slope indicates a drop from the starting point to the end of the 10 points whereas a positive indicates an increase from the starting point to the end.

The more extreme one or more of the slopes, and the number of extreme slopes, compared to normal, the higher probability there is that a cow is lame.

TABLE 2
Mean and standard deviation in mm of slope1 to slope9 in the five herds

| | | | slope | slope | slope | slope | slope | slope | slope | slope | slope |
|---|---|---|---|---|---|---|---|---|---|---|---|
| herd | FREQ | STAT | 1 | 2 | 3 | 4 | 5 | 6 | 7 | 8 | 9 |
| 1 | 35810 | MEAN | −14.03 | −10.06 | −10.62 | 1.54 | −5.74 | −5.68 | −13.96 | −19.57 | −34.99 |
| 1 | 35810 | STD | 18.91 | 16.50 | 12.99 | 11.41 | 13.92 | 16.35 | 17.53 | 18.03 | 19.85 |
| 2 | 21597 | MEAN | −17.64 | −11.23 | −6.18 | 2.59 | −7.38 | −10.37 | −21.98 | −34.73 | −35.94 |
| 2 | 21597 | STD | 19.96 | 18.43 | 16.14 | 14.71 | 14.79 | 15.15 | 16.06 | 17.18 | 18.14 |
| 3 | 9029 | MEAN | −7.76 | 3.42 | 5.70 | 9.17 | 4.33 | −2.38 | −14.26 | −21.80 | −28.34 |
| 3 | 9029 | STD | 19.77 | 17.36 | 14.70 | 13.77 | 14.04 | 17.29 | 20.34 | 22.39 | 25.48 |
| 4 | 41459 | MEAN | −1.51 | 8.54 | 7.76 | 6.99 | −0.82 | −8.64 | −19.64 | −27.30 | −35.18 |
| 4 | 41459 | STD | 19.50 | 18.15 | 14.85 | 11.78 | 13.00 | 16.21 | 18.75 | 20.49 | 22.67 |
| 5 | 24115 | MEAN | −8.27 | −0.95 | 1.52 | 3.09 | −3.30 | −13.00 | −26.23 | −34.20 | −49.48 |
| 5 | 24115 | STD | 20.45 | 18.16 | 16.05 | 15.44 | 17.01 | 18.98 | 20.16 | 22.18 | 26.67 |

In table 2 the mean slope and the standard deviation for each slope is presented for the five herds. In general, slope 1 to slope 3 is negative, slope 4 and 5 are positive and slope 6 to slope 9 is again negative. This indicates that the cows in general have curved backs. The standard deviation are smallest in slope 4 and 5 which indicates that cows are more evenly curved in the middle section of the back compared to rear part and front part of the back. Also the large standard deviations indicate that there in general are very large differences between cows in relation to the curvature of the back.

TABLE 3
Correlation between Slope 1 to slope 9 across all five herds

| | slope1 | slope2 | slope3 | slope4 | slope5 | slope6 | slope7 | slope8 | slope9 |
|---|---|---|---|---|---|---|---|---|---|
| slope1 | 1 | 0.904 | 0.73 | 0.39 | 0.03 | −0.18 | −0.27 | −0.29 | −0.29 |
| slope2 | | 1 | 0.89 | 0.58 | 0.18 | −0.10 | −0.22 | −0.26 | −0.24 |
| slope3 | | | 1 | 0.81 | 0.43 | 0.07 | −0.10 | −0.18 | −0.18 |
| slope4 | | | | 1 | 0.80 | 0.49 | 0.23 | 0.10 | −0.01 |
| slope5 | | | | | 1 | 0.86 | 0.65 | 0.43 | 0.26 |
| slope6 | | | | | | 1 | 0.90 | 0.73 | 0.46 |
| slope7 | | | | | | | 1 | 0.90 | 0.66 |
| slope8 | | | | | | | | 1 | 0.82 |
| slope9 | | | | | | | | | 1 |

Table 3 is showing the correlation between the nine slopes. The correlation is always high between neighboring slopes. Also the correlation between Slope1 to slope 3 with slop 7 to slope 9 is actually negative indicating that the curvature of the front of the cow often is not related and often negatively related to the curvature of the rear end of the cow.

TABLE 4
Animal variation, residual variation, total variation
and repeatability of slope 1 to slope 9 across herds.

| | Animal var. | Residual var. | Total var. | Repeatability |
|---|---|---|---|---|
| Slope1 | 167 | 224 | 391 | 0.43 |
| Slope2 | 142 | 171 | 313 | 0.45 |
| Slope3 | 99 | 118 | 217 | 0.46 |
| Slope4 | 85 | 87 | 172 | 0.49 |
| Slope5 | 107 | 103 | 210 | 0.51 |
| Slope6 | 125 | 160 | 285 | 0.44 |
| Slope7 | 149 | 202 | 351 | 0.42 |
| Slope8 | 185 | 225 | 410 | 0.45 |
| Slope9 | 258 | 260 | 518 | 0.50 |

Table 4 is showing the bovine subject variation, residual variation, and total variation in the slopes from 1 to 9 as well as the repeatability which is defined as the bovine subject variation divided by the total variation. A repeatability close to 0 would indicate that there is no relationship between two measures of the same cow, whereas a repeatability of 1 would indicate that exactly the same is measured every time a cow is registered. In this example repeatabilities between 0.42 and 0.51 are observed. This indicates that there is substantially information that is related to a specific cow in the registration and that this could be used to predict lameness.

An objective, but rought, lameness threshold is the slope of the rear part of the spine, i.e. the slope of the spine from the tail to the middle of the spine. Based on the pictures in FIG. 1 the slope between the height of the spine (point1) and the height at the middle of the spine (point50) were calculated for all cows. A lameness threshold value was defined to be 30 for this slope corresponding to a height difference of 7.5 cm of the spine over the rear part of the back. This is summarized in in table 5 below.

Table 5 shows the frequency of images with
high curvature of a rear part of spine

| | Cows with high slope | Total number of | |
|---|---|---|---|
| Herd | of rear part of spine | images | Frequency in % |
| total | 664 | 132010 | 0.93 |
| 1 | 163 | 35810 | 0.68 |
| 2 | 418 | 21597 | 2.49 |
| 3 | 6 | 9029 | 0.18 |
| 4 | 94 | 41459 | 0.69 |
| 5 | 61 | 24115 | 0.45 |

I.e. around 1% of the cows have a rear part of the spine with a high slope, and can be considered to be lame. This is an example of an global reference threshold value for objectively detecting lameness in bovine subject. However, it is also an example of a very rough threshold value that discard most of the information of the topology of the back that is available for the specific cow.

### Example 2

Sequences of range images from more than 1000 dairy cows from five different herds will be acquired over a time period. Sequences of 3D images of each cows walking in or through the passage can then be generated. The herds are also supervised manually as normal, and some of the cows among the imaged cows will eventually be observed to be lame. This information can be fed into the data for creation of a trained model. Modelling the acquired image data with the trained model might reveal additional cows suspected to be lame, and these cows can be examined further, i.e. the trained model can be continuously improved by constantly feeding new verified lameness data into the model. At some point the model is good enough to be trained on very early data, i.e. on data acquired months before lameness is detected in the cow. In that way the presently disclosed approach can be used for early prediction of lameness.

### REFERENCES

Christen A.-M., C. Egger-Danner, N. Capion, N, Charfeddine, J. Cole, G. Cramer, A. Fiedler, T. Fjeldaas, N. Gengler, M. Haskell, B. Heringstad, M. Holzhauer, G. de Jong, A. Koeck, J. Kofler, K. Müller, J. Pryce, Å. M. Sogstad, K. F. Stock, G. Thomas and E. Vasseur 2021 Recording Lameness in Dairy Cattle. 2021 ICAR meeting

### Items

1. A method for detecting lameness in bovine subjects, the method comprising the steps of
   acquiring at least one 3D image of the back of a bovine subject, and
   extracting data representing the spine of the bovine subject,
   determining at least one curvature of at least one preselected part of the spine, and
   detecting lameness and/or determining a degree of lameness of the bovine subject by correlating said curvature(s) with at least one reference value, such as at least one predefined threshold value.
and/or
   extracting data in the form of contour points, from said at least one 3D image, relating to the topology of the back of the bovine subject,
   detecting lameness and/or determining a degree of lameness of the bovine subject by correlating said contour points against a reference model.
2. The method according item 1, wherein the extracted data comprise at least 50 data points, preferably at least 100 data points.
3. The method according to any preceding items, wherein the reference model comprises information of the topology of the back versus the degree of lameness, for example in relation to the breed of said bovine subject.
4. The method according to any preceding items, wherein the reference value(s) and/or the reference model is specific to said bovine subject.
5. The method according to any preceding items, wherein the reference value(s) and/or the reference model is specific to the breed of said bovine subject.
6. The method according to any preceding items, wherein the reference value(s) and/or the reference model is specific to said bovine subject and generated from previous 3D images of the back of said bovine subject.
7. The method according to any preceding items 6, wherein the previous 3D images of said bovine subject are acquired over a time period of at least two days, more preferably at least five days, even more preferably at least one week, yet more preferably at least two weeks, most preferably at least four weeks.
8. The method according to any preceding items, wherein the curvature of a preselected part of the spine is defined as only the vertical curvature of said preselected part of the spine.
9. The method according to any preceding items, wherein the curvature of a preselected part of the spine is determined by calculating the slope of said preselected part of the spine.
10. The method according to any preceding items, wherein the at least one preselected part of the spine is a rear part of the spine (towards the tail of the bovine subject).
11. The method according to any preceding items, wherein at least part of the spine is divided into subsections, and wherein the curvature of each subsection is determined.
12. The method according to any preceding items 11, wherein at least part of the spine is divided into at least 2, 3, 5, 7, or at least 10 subsections.
13. The method according to any preceding items 11-12, wherein a reference value is provided for each subsection.
14. The method according to any preceding items, wherein detection of lameness correlates with a predefined difference between the curvature(s) and the corresponding reference value.
15. The method according to any preceding items, wherein the degree of lameness correlates with the difference between the curvature(s) and the corresponding reference value.
16. The method according to any preceding items, wherein the contour points are extracted relative to a line of local maxima along the back of the animal.
17. The method according to any preceding items, wherein the spine is defined as a through-going line of local maxima.
18. The method according to any preceding items, comprising a step of identifying the bovine subject based on said at least one 3D image.
19. The method according to any preceding items, comprising a step of identifying the bovine subject based on RFID technology.
20. The method according to any preceding items, wherein the bovine subject is moving during acquisition of said at least one 3D image.
21. The method according to any preceding items, wherein said at least one 3D image is based on a plurality of 3D images acquired while the bovine is moving.
22. The method according to any preceding item, wherein more than one 3D image are acquired of the bovine subject.
23. The method according to any preceding items, wherein said at least one 3D image is based on a plurality of 3D images acquired while the bovine subject is moving.
24. The method according to any preceding items, wherein the data analysis is based on a median image of two or more images.
25. The method according any preceding items, wherein the bovine subject include both cows and bulls, whether adult or newborn animals.
26. The method according any preceding items, wherein the threshold is defined based on the breed of the bovine subject, which breed is selected from the group of: the Jersey breed, Friesian cattle population, Holstein Swartbont cattle population, the Deutsche Holstein Schwarzbunt cattle population, the US Holstein cattle population, the Red and White Holstein breed, the Deutsche Holstein Schwarzbunt cattle population, the Danish Red population, the Finnish Ayrshire population, the Swedish Red and White population, the Danish Holstein population, the Swedish Red and White population and the Nordic Red population.
27. A system for detecting lameness in bovine subjects, comprising:
   an imaging system configured to acquire at least one 3D image of the back of a bovine subject,
   a processing unit configured for
      acquiring at least one 3D image of the back of the bovine subject,
      extracting data representing the spine of the bovine subject,
      determining at least one curvature of at least one preselected part of the spine, and
      detecting lameness of the bovine subject by correlating said curvature(s) against at least one predefined threshold value.
28. The system according to item 27, configured to acquire said at least one 3D image while the bovine subject is standing in and/or walking through a lock.
29. The system according to any of items 27 to 28, configured such that said at least one 3D image is acquired from above the bovine subject thereby imaging said bovine subjects in a top-view.
30. The system according to any of items 27 to 29, configured to acquire said at least one 3D image when triggered by said bovine subject approaching and/or entering a lock.
31. The system according to any of items 27 to 30, wherein the processing unit is configured to execute the method of any of items 1-26.

1. A method for detecting lameness in a bovine subject, the method comprising the steps of
acquiring a plurality of range images of the back of at least one bovine subject, wherein the range images are acquired from above the at least one bovine subject,
image processing the range images to generate at least one sequence of 3D images of a preselected bovine subject, and
detecting lameness and/or determining a degree of lameness of the preselected bovine subject by modelling the at least one sequence of 3D images against a trained reference model employing artificial intelligence (AI).

2. The method according to claim 1, wherein the plurality of range images is a time series of range images, and/or the sequence of 3D images of the preselected bovine subject is a time series of 3D images.

3. The method according to any preceding claims, comprising the step of labelling the preselected bovine subject as “not lame”, “possibly lame” or “moderately lame”, “lame” or “severely lame” after modelling.

4. The method according to any preceding claims, comprising the step of labelling the preselected bovine subject “lame” with a degree of lameness as an integer between 1 and 5 after detection of lameness thereby determining a degree of lameness.

5. The method according to any preceding claims, wherein the range images are acquired with a frame rate of at least 20 frames per second (fps), preferably at least 25 fps.

6. The method according to any preceding claims, wherein the sequence of 3D images comprise the preselected bovine subject walking forwardly.

7. The method according to any preceding claims, wherein the sequence of 3D images comprise the preselected bovine subject walking forwardly over a minimum distance of at least 3 meters.

8. The method according to any preceding claims, wherein the sequence of 3D images comprise the preselected bovine subject during a minimum period of at least 3 seconds, preferably at least 5 seconds.

9. The method according to any preceding claims, wherein the range image acquisition includes acquisition of infrared (IR) 2D image data.

10. The method according to any preceding claims, wherein the image processing comprise the step of generating at least one sequence of 2D IR images of the back of the preselected bovine subject corresponding to the at least one sequence of 3D images.

11. The method according to any preceding claims, wherein the range images are acquired from above a passage where through a plurality of bovine subjects are passing through, such as passing through at least once daily.

12. The method according to any preceding claim 11, further comprising the step of identifying the preselected bovine subject by means of at least one ID reader located one or both ends of the passage.

13. The method according to any preceding claims, wherein the image processing comprise background removal to isolate the bovine subject from the surroundings of the range images.

14. The method according to any preceding claims, wherein the image processing comprise correction of perspective of the range images and/or the 3D images.

15. The method according to any preceding claims, wherein the sequence of 3D images of the preselected bovine subject corresponds to temporal (window) selection of the plurality of range images.

16. The method according to any preceding claims, wherein the sequence of 3D images of the preselected bovine subject corresponds to temporal (window) selection of the plurality of range images selected such that only the preselected bovine subject is present in the sequence of 3D images.

17. The method according to any preceding claims, wherein the image processing comprise the step of segmenting at least part of the acquired range images by means of instance segmentation in order to select a bovine subject among the segmented instances and generate said at least one sequence of 3D images of the preselected bovine subject.

18. The method according to claim 17, wherein instance segmentation is provided by means of Mask-RCNN.

19. The method according to any preceding claims 17-18, wherein the segmented bovine instance located closest an ID reader and a corresponding ID reading is associated with an identification of the preselected bovine subject.

20. The method according to any preceding claims, comprising the step of generating a point cloud of the preselected bovine subject based on the range images and the image processing.

21. The method according to any preceding claims, wherein the at least one sequence of 3D images comprise point cloud coordinates representing the preselected bovine subject in the images.

22. The method according to any preceding claims 20-21, comprising the step of adding depth data to the point cloud data and perspective correcting the point cloud data based on range data/depth data in the range images.

23. The method according to any preceding claims, wherein the sequence of 3D images used as input to the AI model comprise 3D point cloud data representing movement of the preselected bovine subject in said sequence.

24. The method according to any preceding claim 23, wherein the sequence of 3D images used as input to the AI model further comprise 2D IR data of the preselected bovine subject.

25. The method according to any preceding claims, wherein the reference model comprises information of the topology of the back a bovine subjects versus the degree of lameness, for example in relation to the breed of said preselected bovine subject.

26. The method according to any of preceding claims, wherein the trained reference model is trained with a degree of lameness as outcome.

27. The method according to any of preceding claims, wherein the trained model is trained using a supervised learning approach, wherein the AI model is trained using labeled data and wherein the labeled data are obtained from expert veterinarians determining the degree of lameness of bovine subjects determined to be lame by the expert veterinarians.

28. The method according to any of preceding claims, wherein the trained reference model is trained to include a spatial correlation of the lameness in the sequence of 3D images and a temporal correlation of the lameness in the sequence of 3D images.

29. The method according to any of preceding claims, wherein the trained reference model is trained using a combination of self-supervised learning and semi-supervised learning, wherein self-supervised learning approach is provided to pretrain the AI model on unlabeled data, and then the pretrained AI model is fine-tuned on a smaller set of labeled data using semi-supervised learning.

30. The method according to any of preceding claims, wherein the trained reference model is trained with detection of lameness as outcome, and wherein the training data is acquired a period of time before the lameness is detected as outcome and verified manually by an expert.

31. The method according to claim 30, wherein said period of time is at least one months, or at least two months, or at least three months or at least four months.

32. The method according to any of preceding claims, wherein the trained reference model is trained using machine learning, neural network, recurrent neural network, convolutional neural network, or any combination thereof.

33. The method according to any of preceding claims, wherein the AI model is selected from the group of: machine learning, neural network, recurrent neural network, convolutional neural network, or any combination thereof.

34. The method according to any preceding claims, wherein is trained reference model is specific to said preselected bovine subject.

35. The method according to any of preceding claims, wherein the modelling include both spatial correlation and a temporal correlation of lameness in the sequence of 3D images with the trained reference model.

36. The method according to 35, wherein sequence prediction model is multiscale 3D Resnet or ViViT.

37. The method according to any preceding claims, wherein the reference value(s) and/or the reference model is specific to the breed of said bovine subject.

38. The method according to any preceding claims, comprising a step of identifying the bovine subject based on at least one 3D image.

39. The method according to any preceding claims, comprising a step of identifying the bovine subject based on RFID technology.

40. The method according any preceding claims, wherein the breed of the bovine subject is selected from the group of: the Jersey breed, Friesian cattle population, Holstein Swartbont cattle population, the Deutsche Holstein Schwarzbunt cattle population, the US Holstein cattle population, the Red and White Holstein breed, the Deutsche Holstein Schwarzbunt cattle population, the Danish Red population, the Finnish Ayrshire population, the Swedish Red and White population, the Danish Holstein population, the Swedish Red and White population and the Nordic Red population.

41. A system for detecting lameness in bovine subjects, comprising:
an imaging system configured to acquire a plurality of range images of the back of a bovine subject, and
a processing unit configured for executing any of the preceding claims.

42. The system according to claim 41, configured to acquire said range images while the bovine subject is standing in and/or walking through a lock and/or passage

43. The system according to any of claims 41 to 42, configured such that said range images are acquired from above the bovine subject thereby imaging said bovine subjects in a top-view.

44. The system according to any of claims 41 to 43, configured to start acquisition of the range images when triggered by at least one bovine subject approaching and/or entering a lock.

45. The system according to any of claims 41 to 44, wherein the imaging system is configured to acquire the range images with a frame rate of at least 15 frames per second, preferably at least 20, more preferably at least 25 frames per second.

46. The system according to any of claims 41 to 45, comprising one more ID readers for reading an identification number of a bovine subject entering and/or exiting the lock/passage.

47. The system according to any of claims 41 to 46, wherein the imaging unit comprise at least one RGB camera and a depth sensor, such as an infrared sensor for providing 2D infrared images.
